(19) United States
(12) Patent Application Publication
(10) Pub. No.: US 2020/0115314 A1
(43) Pub. Date: Apr. 16, 2020

(54) METHOD OF PRODUCING PLASTICIZER AND REDUCING ENERGY CONSUMPTION

(71) Applicant: SABIC Global Technologies B.V., Bergen op Zoom (NL)

(72) Inventors: Asiff Apdul SUPAHAN, Bangalore (IN); Atul PANT, Bangalore (IN); Ritesh NANDY, Bangalore (IN); Sanjay KATREKAR, Bangalore (IN); Ravinath MANCHANA, Bangalore (IN)

(21) Appl. No.: 16/604,210

(22) PCT Filed: Mar. 13, 2018
(86) PCT No.: PCT/IB2018/051677

§ 371 Date: Oct. 10, 2019

Related U.S. Application Data
(60) Provisional application No. 62/508,766, filed on May 19, 2017.

(51) Int. Cl. C07C 67/08; C07C 69/80; B01J 31/22

(52) U.S. Cl. CPC C07C 67/08; B01J 31/2208; C07C 69/80; B01J 2531/42

(57) ABSTRACT

Systems and methods for producing a non-phthalate based plasticizer. The systems and methods involve dissolving a carboxylic acid and/or anhydride thereof in an alcohol at a temperature below the melting point of the carboxylic anhydride. An advantage of the method is reduced energy consumption compared to conventional methods that require melting the carboxylic anhydride. Furthermore, the method enables the production of non-phthalate based plasticizer in an existing phthalate based plasticizer production facility with minimal modification, thereby reducing capital expenditure.

### CROSS REFERENCE TO RELATED APPLICATIONS

This application claims the benefit of priority of U.S. Provisional Patent Application No. 62/508,766, filed May 19, 2017, which is hereby incorporated by reference in its entirety.

### FIELD OF INVENTION

The present invention generally relates to plasticizer production. More specifically, the present invention relates to a method of producing non-phthalate based plasticizers using production facilities designed for phthalate based plasticizers.

### BACKGROUND OF THE INVENTION

Plasticizers are chemicals added to rubbers or resins to impart flexibility, durability and/or stretchability. Over the past few decades, phthalate based plasticizers, especially ortho-phthalate esters, dominated the plasticizer market due to their low production costs. However, recent discoveries of health and environmental risks posed by phthalate have led to a plummet in demand for phthalate based plasticizers. Therefore, many existing production facilities for phthalate based plasticizers are currently underutilized.

Efforts have been made to retrofit phthalate based plasticizer production facilities with non-phthalate based plasticizer production equipment to enable manufacturing of non-phthalate based plasticizers in the same facilities. Obstacles for producing non-phthalate based plasticizers in such facilities include high energy demand and/or equipment modification required for melting a carboxylic acid and/or anhydride thereof. Methods are employed to replace the melting system in existing facilities with equipment that can operate safely at melting points of carboxylic acids and/or anhydrides thereof used for non-phthalate based plasticizer production. Other methods include changing the reactor for the esterification reaction in existing phthalate based plasticizer production facilities to accommodate solid carboxylic acids and/or anhydrides. However, these methods require major modifications on existing production plants for phthalate based plasticizers, thereby demanding significant capital investment. Furthermore, none of these methods solve the problem of high energy consumption for melting the carboxylic acid and/or anhydride thereof. Improvements in this field are always desired.

### BRIEF SUMMARY OF THE INVENTION

A method has been discovered for producing non-phthalate based plasticizers in existing phthalate based plasticizer production plants. By dissolving carboxylic acid and/or anhydride thereof in an alcohol at a temperature below the melting point of the carboxylic acid and/or anhydride thereof, a non-phthalate based plasticizer may be produced in facilities designed for phthalate based plasticizer production with minimal capital investment and reduced energy consumption.

Embodiments of the invention include a method for producing a non-phthalate based plasticizer. The method may include dissolving a carboxylic acid and/or anhydride thereof in an alcohol in a first vessel at a temperature below a melting point of the carboxylic acid and/or anhydride thereof, to form a solution. The method may further include flowing the solution to a second vessel that has a catalyst disposed therein. The solution contacts the catalyst at reaction conditions sufficient to form the non-phthalate based plasticizer.

Embodiments of the invention include a method for producing a non-phthalate based plasticizer. The method may include dissolving a carboxylic acid and/or anhydride thereof in an alcohol in a first vessel at a temperature below a melting point of the carboxylic acid and/or anhydride thereof to form a solution. The method may further include flowing the solution to a second vessel that has a catalyst disposed therein. The solution may be contacted with the catalyst at reaction conditions sufficient to form the non-phthalate based plasticizer. The method may further include collecting vapor from the first vessel to a reactor condenser. Further still, the method may include purifying the formed non-phthalate based plasticizer.

Embodiments of the invention include a method for producing trioctyl trimellitate (TOTM) in a plant adapted to produce a non-phthalate based plasticizer. The method may include dissolving trimellitic anhydride (TMA) in 2-ethyl hexanol, in a first vessel, at a temperature in a range of 100° C. to 155° C. to form a solution. The method may further include flowing the solution to a second vessel that has a catalyst disposed therein. Further still, the method may include contacting the solution with the catalyst at reaction conditions sufficient to form the TOTM. The reaction conditions may include a temperature in a range of 190° C. to 230° C.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

In the context of the present invention, twenty embodiments are now described. Embodiment 1 is a method for producing a non-phthalate based plasticizer. The method of embodiment 1 includes the steps of dissolving at least one of a carboxylic acid or an anhydride of a carboxylic acid in an alcohol, in a first vessel, at a temperature below a melting point of the carboxylic acid and/or anhydride thereof, to form a solution; flowing the solution to a second vessel that has a catalyst disposed therein; and contacting the solution with the catalyst at reaction conditions sufficient to form the non-phthalate based plasticizer. Embodiment 2 is the method of embodiment 1, wherein the non-phthalate based plasticizer comprises at least one member selected from the group consisting of trioctyl trimellitate (TOTM), trimethyl trimellitate (TMTM), tri-(n-octyl, n-decyl) trimellitate (ATM), n-octyl trimellitate (OTM) and dioctyl terephthalate. Embodiment 3 is the method of embodiment 1, wherein the carboxylic acid includes at least one member selected from the group consisting of trimellitic acid, terephthalic acid, adipic acid and benzoic acid. Embodiment 4 is the method of any of embodiments 1 to 3, wherein the alcohol comprises at least one member selected from the group consisting of 2-ethyl hexanol, methanol, n-octanol and n-decanol. Embodiment 5 is the method of any of embodiments 1 to 4, wherein the temperature in the first vessel during the dissolving is in a range of 110° C. to 160° C. Embodiment 6 is the method of embodiment 5, wherein the temperature in the first vessel during the dissolving is below a design limit temperature of a melter of a dioctyl phthalate production facility. Embodiment 7 is the method of any of embodiments 1 to 6, wherein a molar ratio of the alcohol to the carboxylic acid and/or anhydride thereof in the dissolving is 1:1 to 4:1. Embodiment 8 is the method of any of embodiments 1 to 7, wherein the first vessel is a melter of a dioctyl phthalate production facility for melting solid phthalic anhydride therein. Embodiment 9 is the method of embodiment 8, wherein the first vessel is a melter of the dioctyl phthalate production facility modified by adding a connecting pathway from a fume hood of the melter to a reactor condenser of the dioctyl phthalate production facility. Embodiment 10 is the method of embodiment 9, further including the steps of collecting vapor from the first vessel to the reactor condenser via the connecting pathway. Embodiment 11 is the method of any of embodiments 1 to 10, wherein the second vessel is an esterification reactor of a dioctyl phthalate production facility. Embodiment 12 is the method of any of embodiments 1 to 11, wherein the reaction conditions of the contacting include a temperature of 190° C. to 230° C. Embodiment 13 is the method of any of embodiments 1 to 12, wherein the catalyst contains a protonic acid. Embodiment 14 is the method of embodiment 13, wherein the protonic acid contains at least one member selected from the group consisting of sulfuric acid, methanesulfonic acid, p-toluenesulfonic acid and an alkoxy titanate. Embodiment 15 is the method of embodiment 14, wherein the protonic acid contains at least one alkoxy titanate selected from the group consisting of tetraisopropyl titanate and tetrabutyl titanate. Embodiment 16 is the method of any of embodiments 1 to 15, wherein the carboxylic acid and/or anhydride thereof starts reacting with the alcohol in an esterification reaction in the first vessel. Embodiment 17 is the method of embodiment 16, wherein the esterification reaction completes in the second vessel. Embodiment 18 is the method of any of embodiments 1 to 17, further including the step of purifying the non-phthalate based plasticizer. Embodiment 19 is the method of embodiment 18, wherein the purifying is selected from the group consisting of washing, stripping of alcohol, dewatering, decoloring, filtering and combinations thereof. Embodiment 20 is the method of embodiment 19, wherein the alkoxy titanate contains at least one member selected from the group consisting of tetraisopropyl titanate and tetrabutyl titanate.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a schematic diagram of a non-phthalate based plasticizer production system, according to embodiments of the invention; and

FIG. 2 shows a schematic flowchart for a method of producing a non-phthalate based plasticizer, according to embodiments of the invention.

### DETAILED DESCRIPTION OF THE INVENTION

A method has been discovered for producing a non-phthalate based plasticizer using a production facility designed for a phthalate based plasticizer. Instead of melting carboxylic acid and/or anhydride thereof according to conventional methods, the method includes dissolving a carboxylic acid and/or anhydride thereof in an alcohol below the melting point of the carboxylic acid and/or anhydride thereof, which thereby reduces the energy consumption for producing the non-phthalate based plasticizer. Furthermore, by using embodiments of the invention, the non-phthalate based plasticizer may be produced within the designed temperature limit of the phthalate based plasticizer production facility with minimal modification, thereby minimizing capital expenditure.

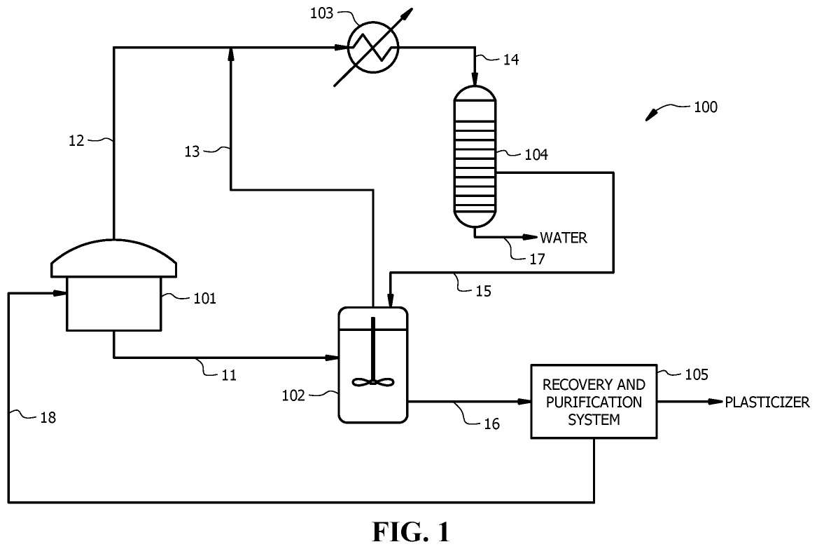

FIG. 1 shows a schematic diagram of system 100 for producing a non-phthalate based plasticizer using a phthalate based plasticizer production facility. The modified phthalate based plasticizer production facility may comprise modified melter 101, esterification reactor 102, reactor condenser 103 configured to condense vapor from esterification reactor 102, phase separator 104 in fluid communication with esterification reactor 102 and reactor condenser 103. System 100 may further comprise recovery and purification system 105 configured to recover and purify the non-phthalate based plasticizer. In embodiments of the invention, modified melter 101 may comprise a container, a fume hood disposed over the container, and a connecting pipe in fluid communication with the fume hood and reactor condenser 103. The connecting pipe of modified melter 101 may be the only modification on the phthalate based plasticizer production facility, according to embodiments of the invention. The melter may have a design upper limit for operating temperature of about 150° C. to about 170° C.

In embodiments of the invention, modified melter 101 may be in fluid communication with esterification reactor 102. An inlet of reactor condenser 103 is in fluid communication with esterification reactor 102. An outlet of reactor condenser 103 is in fluid communication with phase separator 104. An outlet of phase separator 104 is in fluid communication with esterification reactor 102. Esterification reactor 102 may be in fluid communication with recovery and purification system 105. In embodiments of the invention, recovery and purification system 105 may comprise a blender, a filtration system, a distillation system, or combinations thereof. Exemplary esterification reactors 102 may include, but are not limited to a batch reactor, a continuous stirred tank reactor or a semi-continuous reactor. According to embodiments of the invention, the modified phthalate based plasticizer production facility may produce phthalate based plasticizer when the pipe in fluid communication with both the fume hood of modified melter 101 and reactor condenser 103 is closed.

In a conventional phthalate based plasticizer production process, phthalic anhydride is melted in a non-modified melter, and molten phthalic anhydride is pumped to esterification reactor 102 to mix with an alcohol in the presence of catalyst. Phthalic anhydride has a melting point of 130° C., and the non-modified melter may operate at a temperature of about 150° C. However, carboxylic anhydrides used for non-phthalate based plasticizer production, such as trimellitic anhydride, may have a melting point higher than 150° C. and/or higher than the design upper limit for operating temperature of melter 101. Retrofitting existing phthalate based plasticizer facilities with non-phthalate based plasticizer production equipment typically requires replacing the non-modified melter, and/or replacing the esterification reactor to accommodate solid carboxylic anhydrides therein. However, both of these solutions demand high capital expenditure and/or high energy consumption. The method, according to embodiments of the invention, may remedy these deficiencies.

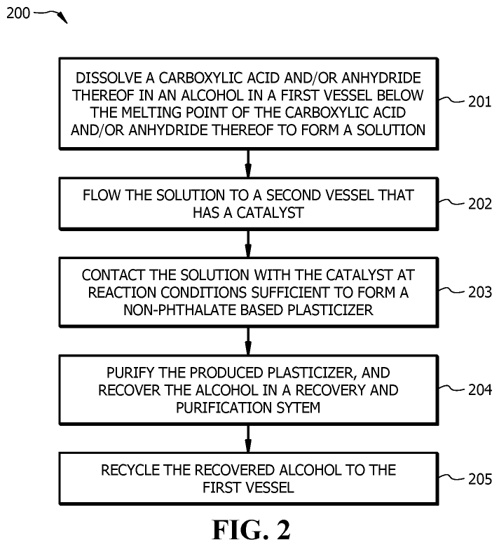

As shown in FIG. 2, embodiments of the invention include method 200 for producing a non-phthalate based plasticizer. Method 200 may be performed in a modified phthalate based plasticizer production facility. For example, the phthalate based plasticizer production facility may be a production plant for dioctyl phthalate. The modification may be a connecting pipe and/or pathway between a fume hood of melter 101 and reactor condenser 103, as described above. The non-phthalate based plasticizer may include trioctyl trimellitate (TOTM), trimethyl trimellitate (TMTM), tri-(n-octyl, n-decyl) trimellitate (ATM), n-octyl trimellitate (OTM), dioctyl terephthalate, or combinations thereof.

As shown in block 201, a carboxylic acid and/or anhydride thereof may be dissolved in an alcohol in a first vessel at a temperature below a melting point of the carboxylic acid and/or anhydride thereof to form a solution. The carboxylic acid may include trimellitic acid, terephthalic acid, adipic acid, benzoic acid, or combinations thereof. The alcohol may include 2-ethyl hexanol, methanol, n-octanol, n-decanol, or combinations thereof. The molar ratio of the alcohol to the carboxylic acid and/or anhydride thereof may be from 1 to 4 and all ranges and values there between including 1 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.1, 2.1 to 2.2, 2.2 to 2.3, 2.3 to 2.4, 2.4 to 2.5, 2.5 to 2.6, 2.6 to 2.7, 2.7 to 2.8, 2.8 to 2.9, 2.9 to 3.0, 3.0 to 3.1, 3.1 to 3.2, 3.2 to 3.3, 3.3 to 3.4, 3.4 to 3.5, 3.5 to 3.6, 3.6 to 3.7, 3.7 to 3.8, 3.8 to 3.9, or 3.9 to 4.0.

In embodiments of the invention, the temperature below the melting point of the carboxylic acid and/or anhydride thereof may be a temperature below 164° C. According to embodiments of the invention, the temperature in the first vessel for the carboxylic acid and/or anhydride thereof dissolving in the alcohol may be in a range of 110° C. to 160° C. For instance, the temperature for trimellitic acid dissolving in 2-ethyl hexanol may be about 110° C. to about 150° C. and all values and ranges there between including 110° C. to 112° C., 112° C. to 114° C., 114° C. to 116° C., 116° C. to 118° C., 118° C. to 120° C., 120° C. to 122° C., 122° C. to 124° C., 124° C. to 126° C., 126° C. to 128° C., 128° C. to 130° C., 130° C. to 132° C., 132° C. to 134° C., 134° C. to 136° C., 136° C. to 138° C., 138° C. to 140° C., 140° C. to 142° C., 142° C. to 144° C., 144° C. to 146° C., 146° C. to 148° C., or 148° C. to 150° C. Since energy required for dissolving the carboxylic acid and/or anhydride thereof in the alcohol is much lower than energy required for melting the carboxylic acid and/or anhydride thereof, method 200 may reduce the operating costs and avoid the capital costs needed for replacing the melter or esterification reactor.

In embodiments of the invention, the temperature below the melting point of the carboxylic acid and/or anhydride thereof in block 201 is not higher than a temperature of a design limit of the first vessel. For example, in the case of trioctyl trimellitate production in a dioctyl phthalate production facility with modified melter 101, the melter of the dioctyl phthalate production facility does not provide a wide enough temperature range to process molten trimellitic anhydride safely since trimellitic anhydride has a melting point of about 164° C. Dissolving the carboxylic acid and/or anhydride thereof in the alcohol below the melting point, as shown in block 201 remedies this situation.

In embodiments of the invention, the esterification reaction between the carboxylic acid and/or anhydride thereof may start in the first vessel, forming water and the non-phthalate based plasticizer. The first vessel may be modified melter 101. The esterification reaction in the first vessel may be monoesterification and/or diesterification. Vapor in stream 12 comprising the formed water during esterification and the alcohol may be transferred to reactor condenser 103 by the fume hood of melter 101. In block 202, the solution formed in block 201 comprising the formed water, the alcohol, the plasticizer, the carboxylic acid and/or anhydride thereof may form stream 11, which may be flowed to a second vessel. The second vessel may be esterification reactor 102. A catalyst may be disposed in the second vessel.

In embodiments of the invention, the catalyst may comprise a protonic acid. Exemplary protonic acids may include, but are not limited to, sulfuric acid, methanesulfonic acid, p-toluenesulfonic acid, an alkoxy titanate, or combinations thereof. Examples for alkoxy titanate may include tetraisopropyl titanate, tetrabutyl titanate, or combinations thereof. As shown in block 203, the solution flowed to the second vessel (esterification reactor 102) may be contacted with the catalyst at reaction conditions sufficient to form the non-phthalate based plasticizer. Upon the contact between the solution and the catalyst, agitation and heating may be applied to the second vessel. In block 203, the molar ratio of the alcohol to the carboxylic acid and/or anhydride in the second vessel may be greater than that in the first vessel to provide excess alcohol for the esterification reaction. The reaction in the second vessel may be a monoesterification, diesterification, and/or triesterification reaction. The reaction conditions may comprise a reaction temperature of 190° C. to 230° C. and all ranges and values there between including ranges of 190° C. to 192° C., 192° C. to 194° C., 194° C. to 196° C., 196° C. to 198° C., 198° C. to 200° C., 200° C. to 202° C., 202° C. to 204° C., 204° C. to 206° C., 206° C. to 208° C., 208° C. to 210° C., 210° C. to 212° C., 212° C. to 214° C., 214° C. to 216° C., 216° C. to 218° C., 218° C. to 220° C., 220° C. to 222° C., 222° C. to 224° C., 224° C. to 226° C., 226° C. to 228° C., or 228° C. to 230° C.

Vapor generated in the second vessel (esterification reactor 102) comprising primarily water and alcohol may form stream 13 and enter reactor condenser 103. Vapor of stream 12 and stream 13 may be condensed in reactor condenser 103 to form a condensate of stream 14. The condensate of stream 14 in turn flows into phase separator 104 to form stream 15 comprising primarily the alcohol and stream 17 comprising primarily water. The alcohol in stream 15 may be recycled back to the second vessel (esterification reactor 102). In embodiments of the invention, substantially all the formed water during esterification reaction in the second vessel (esterification reactor 102) may be removed via reactor condenser 103 and phase separator 104. Under the conditions of excess alcohol in the second vessel (esterification reactor 102), the reaction temperature of 190° C. to 230° C., and adequate water removal via phase separator 104, a high conversion rate of the carboxylic acid and/or anhydride thereof may be achieved. The conversion rate of the carboxylic acid and/or anhydride thereof, according to embodiments of the invention, may be 95% to 100% and all ranges and values there between, including ranges of 95.0% to 95.5%, 95.5 to 96.0%, 96.0% to 96.5%, 96.5% to 97.0%, 97.0% to 97.5%, 97.5% to 98.0%, 98.0% to 98.5%, 98.5% to 99.0%, 99.0% to 99.5%, or 99.5% to 100%.

First product stream 16 then flows to recovery and purification system 105. First product stream 16 may comprise the non-phthalate based plasticizer, water, the alcohol, and/or the catalyst. As shown in block 204, the produced non-phthalate based plasticizer in first product stream 16 may be purified in recovery and purification system 105. In embodiments of the invention, the purifying process may comprise washing, stripping of alcohol, filtration, dewatering, decoloring, or combinations thereof. The purified non-phthalate based plasticizer may have a color value of less than 30 under standards of American Public Health Association (APHA). The alcohol from stream 16 may be recovered via recovery and purification system 105. Block 205 shows that the recovered alcohol may form stream 18 and be recycled back to the first vessel. In embodiments of the invention, method 200 including blocks 201 to 205 may be a batch process, a continuous process or a semi-continuous process.

To sum up, embodiments of the invention involve a method for producing non-phthalate based plasticizer. The method may comprise dissolving a carboxylic acid and/or anhydride thereof in an alcohol at a temperature below a melting point of the carboxylic acid and/or anhydride thereof to form a solution, and pumping the solution to the reactor for esterification. Therefore, the method may be performed under the design temperature limit of the phthalate based plasticizer production facility. The only modification on the phthalate based plasticizer production facility that may be required, according to embodiments of the invention, is adding a connecting pathway between the fume hood of the melter to the reactor condenser of the phthalate based plasticizer production facility, thereby minimizing the capital investment required for facility modification.

Although embodiments of the present invention have been described with reference to blocks of FIG. 2, it should be appreciated that operation of the present invention is not limited to the particular blocks and/or the particular order of the blocks illustrated in FIG. 2. Accordingly, embodiments of the invention may provide functionality as described herein using various blocks in a sequence different than that of FIG. 2.

As part of the disclosure of the present invention, a specific example is included below. The example is for illustrative purposes only and is not intended to limit the invention. Those of ordinary skill in the art will readily recognize parameters that can be changed or modified to yield essentially the same results.

### EXAMPLE

### Solubility Tests for Trimellitic Anhydride in 2-Ethyl Hexanol

Tri octyl trimellitate (TOTM) is one of the most commonly used non-phthalate based plasticizers. Tri octyl trimellitate is produced via esterification reaction between trimellitic anhydride (TMA) and 2-ethyl hexanol (2-EH). According to embodiments of the invention, trimellitic anhydride may be dissolved in 2-ethyl hexanol before it is transferred to an esterification reactor. Thus, the solubility of trimellitic anhydride in 2-ethyl hexanol was tested under various molar ratios of trimellitic anhydride to 2-ethyl hexanol. In this experiment, a hundred grams of trimellitic anhydride was initially added to 2-ethyl hexanol at a molar ratio of 1:1 to form a mixture. The temperature of the mixture was controlled in a range of 110° C. to 150 ° C. Additional 2-ethyl hexanol was gradually added to the mixture to change the molar ratio of trimellitic anhydride to 2-ethyl hexanol until complete dissolution of trimellitic anhydride in 2-ethyl hexanol was observed. The amount of added 2-ethyl hexanol was recorded to obtain a molar ratio needed for the complete dissolution of trimellitic anhydride. The results are shown in Table 1.

TABLE 1
Results of solubility tests for trimellitic
anhydride in 2-ethyl hexanol

| SI | TMA:2-EH | Temperature | | |
|---|---|---|---|---|
| No. | (molar ratio) | (° C.) | Dissolution | Comments |
| 1 | 1:1 | 110-150 | No | Water formation starts |
| | | | | at 135° C. |
| 2 | 1:2 | 110-150 | No | Water formation starts |
| | | | | at 135° C. |
| 3 | 1:2.5 | 110-150 | No | Water formation starts |
| | | | | at 135° C. |
| 4 | 1:3 | 110-150 | No | Water formation starts |
| | | | | at 135° C. |
| 5 | 1:3.25 | 110-150 | Yes | Completely soluble |
| | | | | at 150° C. |

Table 1 shows that a complete dissolution of trimellitic anhydride was observed when the molar ratio of trimellitic anhydride to 2-ethyl hexanol was 1:3.25 at a temperature of about 150° C. At each molar ratio, water formation started at a temperature of 135° C., indicating that esterification reaction between trimellitic anhydride and 2-ethyl hexanol started at 135° C. without presence of catalysts.

The temperature range for complete dissolution of trimellitic anhydride was further repeated by varying the temperature in a range of 110° C. to 149° C. when the molar ratio of trimellitic anhydride to 2-ethyl hexanol was kept at 1:3.25. About 100 g of trimellitic anhydride was used. When a complete dissolution of trimellitic anhydride was observed, the amount of water formed during the dissolution and total time consumed for dissolution are recorded. The results are shown in Table 2.

TABLE 2
Results for repeated solubility test for
trimellitic anhydride in 2-ethyl hexanol

| SI | TMA:2-EH | Temperature | | |
|---|---|---|---|---|
| No. | (molar ratio) | (° C.) | Dissolution | Comments |
| 1 | 1:3.25 | 110-149 | Yes | Water formation starts |
| | | | at 149° C. | at 135° C. |

About 4 ml water is collected when the
mixture is kept for 1 hour at 149° C.
Turbidity starts forming in the solution at
65° C. and the mixture becomes milky at 60° C.

Results in Table 2 confirm that the trimellitic anhydride is completely soluble in 2-ethyl hexanol at a temperature of about 149° C. in 20-30 minutes when the molar ratio of trimellitic anhydride to 2-ethyl hexanol is about 1:3.25. Water formation starting at 135° C. was also confirmed. About 4 ml water was collected when the solution was kept at 149° C. for 1 hour. The cooling process of the solution started when the oil bath used for heating is removed. Turbidity formation was observed in the solution starts at 65° C. The mixture becomes milky when the temperature decreases to 60° C. The turbidity formation indicates that trimellitic acid began to solidify from the solution at 65° C. as the temperature decreases, providing information for safeguarding equipment such as rotary equipment, agitator and pump.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method for producing a non-phthalate based plasticizer, the method comprising:
dissolving a carboxylic acid and/or anhydride thereof in an alcohol, in a first vessel, at a temperature below a melting point of the carboxylic acid and/or anhydride thereof, to form a solution;
flowing the solution to a second vessel that has a catalyst disposed therein; and
contacting the solution with the catalyst at reaction conditions sufficient to form the non-phthalate based plasticizer.

2. The method of claim 1, wherein the non-phthalate based plasticizer is selected from the group consisting of trioctyl trimellitate (TOTM), trimethyl trimellitate (TMTM), tri-(n-octyl, n-decyl) trimellitate (ATM), n-octyl trimellitate (OTM), dioctyl terephthalate, and combinations thereof

3. The method of claim 1, wherein the carboxylic acid is selected from the group consisting of trimellitic acid, terephthalic acid, adipic acid, benzoic acid, and combinations thereof.

4. The method of claim 1, wherein the alcohol is selected from the group consisting of 2-ethyl hexanol, methanol, n-octanol, n-decanol, and combinations thereof.

5. The method of claim 1, wherein the temperature in the first vessel during the dissolving is in a range of 110° C. to 160° C.

6. The method of claim 5, wherein the temperature in the first vessel during the dissolving is below a design limit temperature of a melter of a dioctyl phthalate production facility.

7. The method of claim 1, wherein a molar ratio of the alcohol to the carboxylic acid and/or anhydride thereof in the dissolving is 1:1 to 4:1.

8. The method of claim 1, wherein the first vessel is a melter of a dioctyl phthalate production facility for melting solid phthalic anhydride therein.

9. The method of claim 8, wherein the first vessel is a melter of the dioctyl phthalate production facility modified by adding a connecting pathway from a fume hood of the melter to a reactor condenser of the dioctyl phthalate production facility.

10. The method of claim 9, further comprising collecting vapor from the first vessel to the reactor condenser via the connecting pathway.

11. The method of claim 1, wherein the second vessel is an esterification reactor of a dioctyl phthalate production facility.

12. The method of claim 1, wherein the reaction conditions of the contacting comprise a temperature of 190° C. to 230° C.

13. The method of claim 1, wherein the catalyst comprises a protonic acid.

14. The method of claim 13, wherein the protonic acid is selected from the group consisting of sulfuric acid, methanesulfonic acid, p-toluenesulfonic acid, an alkoxy titanates, and combinations thereof.

15. The method of claim 14, wherein the alkoxy titanates is selected from the group consisting of tetraisopropyl titanate, tetrabutyl titanate, and combinations thereof.

16. The method of claim 1, wherein the carboxylic acid and/or anhydride thereof starts reacting with the alcohol in an esterification reaction in the first vessel.

17. The method of claim 16, wherein the esterification reaction completes in the second vessel.

18. The method of claim 1, further comprising purifying the non-phthalate based plasticizer.

19. The method of claim 18, wherein the purifying is selected from the group consisting of washing, stripping of alcohol, dewatering, decoloring, filtering and combinations thereof.

20. The method of embodiment 19, wherein the alkoxy titanate contains at least one member selected from the group consisting of tetraisopropyl titanate and tetrabutyl titanate.
